(19) United States
(12) Patent Application Publication
(10) Pub. No.: US 2025/0037849 A1
(43) Pub. Date: Jan. 30, 2025

(54) METHOD FOR PREDICTING CONSUMPTION OF SURGICAL CONSUMABLES DURING SURGICAL OPERATION

(71) Applicant: nSight Surgical Inc., San Francisco, CA (US)

(72) Inventors: Nathaniel C. R. Smith, San Francisco, CA (US); Derek Amanatullah, Menlo Park, CA (US); Colin Willson, Alameda, CA (US); Mohammad Haghighat, San Jose, CA (US); Navid Fallahinia, Watertown, MA (US); Noah Davis, Salt Lake City, UT (US); Isaac Yerrington, Bolinas, CA (US); Negash Asegde, San Francisco, CA (US); Justin Bowen, San Francisco, CA (US); J.D. Zamfirescu, Oakland, CA (US)

(21) Appl. No.: 18/786,053

(22) Filed: Jul. 26, 2024

Related U.S. Application Data
(60) Provisional application No. 63/529,117, filed on Jul. 26, 2023.

(51) Int. Cl. G16H 40/20; G06T 7/00

(52) U.S. Cl. CPC G16H 40/20; G06T 7/0012

(57) ABSTRACT

A method includes, at a first time: accessing a first image of a surgical field during a surgical operation; and identifying a first quantity of unused units of a first consumable type in the first image. The method also includes, at a second time: accessing a second image of the surgical field; and identifying a second quantity of unused units of the first consumable type in the second image. The method also includes: predicting a total consumption quantity of the first consumable type during the surgical operation based on a difference between the first quantity and the second quantity; and, in response to the second quantity falling below the total consumption quantity, rendering a prompt on a display, adjacent to an exit of the operating room, to retrieve a set of units of the first consumable type.

### CROSS-REFERENCE TO RELATED APPLICATIONS

This Application claims the benefit of U.S. Provisional Application No. 63/529,117, filed on 26 Jul. 2023, which is incorporated in its entirety by this reference.

### TECHNICAL FIELD

This invention relates generally to the field of surgical operation management and more specifically to a new and useful method for tracking consumables within an operating room in the field of surgical operation management.

### BRIEF DESCRIPTION OF THE FIGURES

FIG. 1 is a flowchart representation of a method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

A method includes, at a first time: accessing a first image of a surgical field during a surgical operation; and identifying a first quantity of unused units of a first consumable type in the first image. The method also includes, at a second time: accessing a second image of the surgical field; and identifying a second quantity of unused units of the first consumable type in the second image. The method also includes: predicting a total consumption quantity of the first consumable type during the surgical operation based on a difference between the first quantity and the second quantity; and, in response to the second quantity falling below the total consumption quantity, rendering a prompt on a display, adjacent to an exit of the operating room, to retrieve a set of units of the first consumable type.

### 1.1 Variation

In one variation, a method includes, at a first time: accessing a first image of a surgical field during a surgical operation; and identifying a first quantity of unused units of a first consumable type in the first image. The method also includes, at a second time: accessing a second image of the surgical field; and identifying a second quantity of unused units of the first consumable type in the second image. The method also includes, at a third time: accessing a third image of the surgical field; detecting an exit of a surgical staff member from the operating room based on the third image; and, in response to detecting exit of the surgical staff member from the operating room, predicting total consumption quantity of the first consumable type during the surgical operation based on a difference between the first quantity and the second quantity. The method also includes, in response to the second quantity falling below the total consumption quantity, rendering a prompt on a display, adjacent to an entrance of operating room, to retrieve a set of units of the first consumable type, based on the difference between the first quantity and the second quantity, prior to reentry into the operating room.

### 1.2 Variation

In one variation, a method includes, at a first time: accessing a first image of a surgical field during a surgical operation; and identifying a first quantity of unused units of a first consumable type in the first image. The method also includes, at a second time: accessing a second image of the surgical field; and identifying a second quantity of unused units of the first consumable type in the second image. The method also includes, at a third time: accessing a third image of the surgical field; and detecting a first exit of a surgical staff member from the operating room based on the third image. The method also includes, at a fourth time: accessing a fourth image of the surgical field; and detecting a second exit of a surgical staff member from the operating room based on the fourth image.

In this variation, the method also includes: predicting a third exit of a surgical staff member from the operating room at a fifth time based on the first exit and the second exit; predicting a consumption quantity of the first consumable type after the fifth time during the surgical operation based on a difference between the first quantity and the second quantity; and, in response to the second quantity falling below the total consumption quantity, rendering a prompt on a display, adjacent an exit of the operating room, to retrieve a set of units of the consumable type, based on the difference between the first quantity and the second quantity, prior to reentry into the operating room.

### 2. System

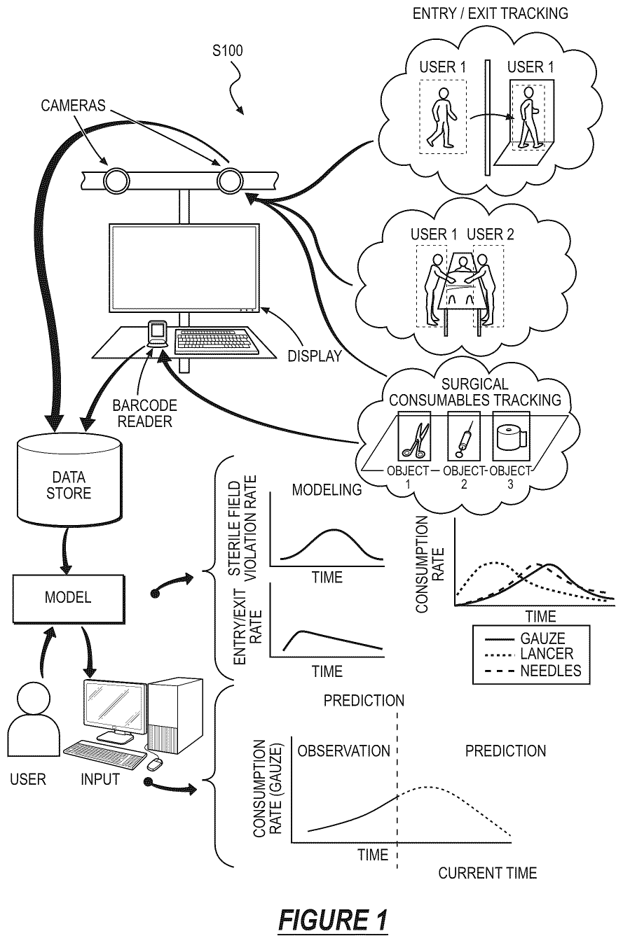

As shown in FIG. 1, a system includes: a set of depth sensors; a set of cameras; a barcode reader; and a computer system.

The set of depth sensors-mounted to overhead locations on a perimeter wall of the operating room-can include a set of cameras. The set of depth sensors can capture depth images and offload these depth images to the computer system to fuse these depth images into low(er) resolution three-dimensional (or “3D”) representations (or “maps”) of the operating room. The set of cameras can include a two-dimensional (or “2D”) pan-tilt-zoom color camera and capture high(er)-resolution images of hands.

The barcode scanner can capture a barcode printed on a surgical consumable, translate the barcode into an identifier (e.g., serial number), and transmit the identifier to the computer system.

### 3. Applications

Generally, the computer system is configured to track real-time use (or “consumption”) of surgical consumables in an operating room during a surgical operation, such as surgical sponges, gauzes, needles, and/or retractors. The computer system is also configured to: predict future consumption of these consumables during the surgical operation based on past and current consumption (e.g., quantities, rates) during this surgical operation and/or other similar surgical operations; predict risk and/or future out-of-stock events for a particular consumable; detect surgical consumables entering and exiting the operating room; and selectively and opportunistically rendering prompts for surgical staff to retrieve more units of a particular consumable during their normal course of entering and exiting the surgical room.

Therefore, the computer system can reduce a total quantity of entry/exit events by combining consumable replenishment actions with unscheduled discretionary staff entry/exit events, thereby reducing infection risk for a patient, which may be correlated with total count of entry/exit events during surgical operation. Furthermore, the computer system can predict future entry/exit events, such as by extrapolating historical and current entry/exit events or based on historical entry/exit event patterns in similar past surgical operations.

In addition, the computer system can present the consumable retrieval prompts to surgical staff: at a time that coincides with likely entry/exit events; or at a time that maximizes delay in retrieval of additional consumables while also avoiding out-of-stock events for a particular consumable within the operating room.

Therefore, the computer system can: enable the surgical staff to prevent out-of-stock events of consumables during a surgical operation; collect additional data on consumption to enable more accurate estimation of consumable count needed for the surgery; and minimize-or avoid altogether-exit/entry events specifically to retrieve more consumables.

Furthermore, the computer system can: enable staff to stock the operating room with a nominal or minimal count of a consumable at the start of a surgery, such as based on average or lower bounds of consumables previously consumed during similar surgical operations; autonomously track consumption of the consumable and surgical staff entering and exiting the operating room via a suite of optical sensors (e.g., color and/or depth sensors) arranged in the operating room; predict future need for additional units of the consumable during the surgical operation; opportunistically prompt staff exiting from or returning to the operating room to retrieve additional units of consumables—such as via a display arranged near the exit and entrance of the operating room—and thus avoid additional entry/exit events solely for consumable restocking. The computer system an also: reduce consumable waste without increasing entry/exit events by generating opportunistic consumable retrieval prompts during current or predicted future entry/exit events; and avoid out-of-stock conditions for the consumable during the surgical operation based on consumption trends during the surgical operation and/or based on consumption trends during similar past surgical operations.

### 4. Entry/Exit Event Tracking

Generally, the computer system can track movement of surgical staff members (e.g., members of surgical staff) in and out of an operating room during a surgical operation. In particular, the computer system can record the number of entry/exit events in the operating room occurring during the surgical operation. Therefore, the computer system can: collect dynamic (i.e., time series) data of entry/exit events; and dynamically track the entry/exit events. In one example, based on the data, the computer system can: calculate the durations of absences of the surgical staff from the operating room (e.g., how long a surgical staff member spent outside the operating room); and based on these durations, estimate a risk (e.g., risk of infection of the patient) associated with the entry/exit events. Therefore, based on this data, the computer system can predict the number of entry/exit events for a particular surgical operation type, operating room, surgical team, and/or patient type.

In one implementation, the computer system can: access a sequence of frames captured by a camera; identify surgical staff members within each frame in the sequence of frames; and execute a classification model trained to classify surgical staff members within a particular operating room in the sequence of frames. The computer system can then track surgical staff members within the operating room across the sequence of frames to: identify entry/exit events; and determine the number of entry/exit events. In particular, the computer system can identify an operating room exit event and increment a count of operating room exit events in response to identifying the first surgical staff member in the first area (e.g., surgical space) in a first frame in the sequence of frames and identifying the first surgical staff member in the second area (e.g., exit area) in a second frame succeeding the first frame.

In one implementation, the computer system can access a first frame of an operating room during a surgical operation, the first frame including a boundary defining the exit area in the operating room. Additionally, or alternatively, the computer system can superimpose a boundary onto the first frame of the operating room such that the boundary is positioned near a door into the operating room in the first image. The computer system can identify an exit event and increment the count of exit events in response to: identifying a first surgical staff member outside the boundary (e.g., surgical space) in a first frame in the sequence of frames; identifying the first surgical staff member inside the boundary (e.g., in the exit area) in a second frame succeeding the first frame; and identifying an absence of the first surgical staff member in a third frame succeeding the second frame.

In one implementation, the computer system can identify an entrance event associated with a first surgical staff member, in the group of surgical staff members, into the operating room in response to: identifying an absence of a first surgical staff member in the operating room in the first frame in the sequence of frames; identifying a first surgical staff member inside the boundary (e.g., inside the exit area) in a second frame succeeding the first frame; and identifying the first surgical staff member outside the boundary (e.g., surgical space) in a third frame succeeding the second frame.

In one implementation, the computer system can identify a surgical staff member entering the operating room as a surgical staff member who has previously been in the operating room. In particular, in response to a first surgical staff member re-entering the operating room, the computer system can classify the surgical staff member re-entering the operating room as the first surgical staff member. Therefore, the computer system can increment a count of entry/exit events representing the total number of entry events and exit events into the operating room in response to identifying an exit of a first surgical staff member from an operating room at the first time and an entrance of the first surgical staff member into the operating room at the second time succeeding the first time.

In one implementation, in response to incrementing the count of total entry/exit events, the count of entry events, or the count of exit events, the computer system can record the count of total entry events and exit events, the count of operating room exits, or the count of operating room entrances and a corresponding timestamp in the dataset associated with the surgical operation. Therefore, the computer system can track the increase of the number of entry/exit events of the surgical staff into and out of the operating room during a surgical operation.

In this implementation, based on the dataset associated with the surgical operation, the computer system can generate a timeseries of the number of entry events and/or exit events into and out of the operating room that occurred during an operation.

In addition, based on an entry/exit vector, the computer system can: identify durations of entry events and exit events, identify durations of entry events, exit events associated with each surgical staff member entering or exiting the operating room, and record the durations of absences of the surgical staff from the operating room. Therefore, the computer system can estimate an increase in risk of the surgical operation based on the durations of durations of absences of the surgical staff from the operating room.

### 5. Consumables Tracking and Recognition

Generally, the computer system can track consumption of surgical consumables during the surgical operation. In particular, the computer system can track a quantity of each type of surgical consumable available on the supply table and quantity of each type of surgical consumable consumed during the surgical operation. Therefore, the computer system can collect dynamic (i.e., time series) data of the number and type of surgical consumables consumed during various types of surgical operations and under various surgical conditions (e.g., type of operating room, surgical staff, patient characteristics). Based on the data, the computer system can predict total number of each type of surgical consumable expected to be consumed during a specific type of surgical operation or predict an out-of-stock event (e.g. shortage) of surgical consumables during the operation.

### 5.1 Camera-Based Tracking of Surgical Consumables

In one variation, the computer system can: access a sequence of frames captured by a second set of cameras and identify a set of consumables within each frame in the sequence of frames; and execute a consumable classification model trained to classify consumables (e.g., surgical consumables such as surgical sutures, bandages, gauze, syringes, tape, etc.) within the sequence of frames. Then, in response to identifying a first consumable in the sequence of frames, the computer system can: classify the first consumable (e.g., surgical consumable) as a first consumable type (e.g., surgical suture); identify a location of the first consumable in the operating room; and, based on the location of the first consumable, identify a status of the first consumable, the status of the first consumable indicating whether the first consumable is available or consumed.

In one implementation, the computer system can, at a first time: access a first image of a surgical field during a surgical operation; identify a first quantity of consumed (e.g., used) units of a first consumable type in the surgical field; and record the first quantity of the first consumable type consumed and the corresponding timestamp in the database associated with the first consumable type. Therefore, the computer system can identify the first quantity of first consumable type consumed at a particular time based on the number of units of the first type of surgical consumable identified in the surgical field at that time.

In one implementation, the computer system can, at a first time: access a first image of a surgical supply table during a surgical operation; identify a first quantity of unused (e.g., available) units of a first consumable type in the first image; and record the first quantity of the first consumable type unused and the corresponding timestamp in the database associated with the first consumable type. Therefore, the computer system can identify the first quantity of first consumable type available (e.g., unused) at a particular time during the surgical operation based on the number of units of the first type of surgical consumable identified in the surgical supply table at that time.

### 5.2 Bar-Code-Based Tracking of Surgical Consumables

In one variation, the computer system can receive an identifier of a first consumable from a barcode scanner and, in response to receiving the identifier (e.g., identification number) of the first consumable: based on the identifier, identify the first consumable as the first consumable type; set a status of the first consumable as consumed; record in a dataset associated with the surgical operation the identifier of the first consumable and the status of the first consumable; record in the dataset the number of units of the first consumable type consumed and the corresponding timestamp. Therefore, the computer can track the number of units of each type of surgical consumable consumed during the surgical operation based on user input such as a barcode scan of each surgical consumable utilized during the surgical operation. For example, a circulating nurse may scan the first surgical consumable (e.g., surgical suture) with the barcode scanner prior to providing the first consumable to the surgeon.

In one implementation, the computer system can train the consumable classification model to classify consumables based on the identifiers received from the barcode scanner and corresponding images received from a set of cameras. In particular, the computer system can simultaneously: access a sequence of frames captured by the set of cameras; and receive an identifier of a first consumable from the barcode scanner. Then, the computer system can: identify an unknown consumable within each frame in a subset of frames in the sequence of frames; label the unknown consumable identified in the subset of frames as the first consumable associated with the identification number received from the barcode; store the subset of frames labeled as a first consumable in a training data store. The computer system can then repeat this process for other consumables and identification numbers received from the barcode scanner. Then, the computer system can train the consumable classification model based on data in the training data store to classify consumables identified in a new sequence of frames (e.g., without receiving an identifier from the barcode scanner).

### 6. Data Aggregation

Generally, the computer system can repeat the process of collecting timeseries data of the number of entrance/exit events; the number and types of surgical consumables available and consumed for various surgical conditions including various of surgeries, various operating rooms, various surgical teams, and various types of patients.

In one implementation, after completion of each surgical operation in a set of surgical operations, the computer system can: generate and store in a database a dataset including: a timeseries of the number of entrance/exit events; and a timeseries of the number and types of surgical consumables available and consumed during the surgical operation. In addition, after completion of each surgical operation in a set of surgical operations, the computer system can tag the dataset with tags indicating surgical conditions associated with the sugary, the surgical conditions including type of surgical operation (e.g., hip arthroplasty, knee arthroplasty), operating room dentification (e.g., operating room number/name), surgical team (e.g., names of surgeon and the surgical staff members present during the operation), and characteristics of the patient (e.g., age, sex, health conditions). Thus, the computer system can generate and store a set of datasets, each dataset in the set of datasets: associated with a unique surgical operation; and tagged with surgical conditions associated with each operation in the set of operations.

In this implementation, the computer system can query the database to retrieve datasets associated with a particular set of surgical conditions. For example, the computer system can query the database for datasets associated with hip arthroplasty surgeries. In another example, the computer system can query the database for datasets associated with surgeries of diabetic patients over 60 years old. Therefore, the computer system can query the database to retrieve datasets associated with a particular set of surgical conditions and, based on the retrieved datasets, predict the number of entrance/exit events expected and/or the quantities and types of surgical consumables needed during a surgical operations associated with the particular set of surgical conditions.

### 7. Modelling

Generally, the computer system can generate a model of consumption of surgical consumables for various surgical conditions (e.g., types of surgeries, patient characteristics, surgical teams, operating rooms) based on the data collected by the computer system during surgical procedures. In particular, the computer system can: identify patterns in the rates of consumption of the surgical consumables; based on the patterns, generate a model of surgical consumable consumption that reflects these patterns; and, based on these patterns, predict consumption of surgical consumables for a given segment of surgical conditions. Therefore, the computer system can predict numbers and types of consumables utilized during a surgical operation defining a set of surgical conditions based on the model.

In particular, the computer system can construct a model quantifying a relationship between any input in a set of inputs characterizing the surgical operation at an earlier time and any output in a set of outputs characterizing the surgical operation at a later time. The set of inputs defined by the model can include: surgical conditions (i.e., surgical operation type, surgical team, operating room, patient characteristics) associated with a surgical operation; numbers of entry/exit events that occurred prior to a first time during surgical operation; rates of entry/exit events recorded at the first time; consumption rates of each type of surgical consumable recorded at the first time; and numbers of each type of surgical consumable available or consumed prior to the first time. The set of outputs defined by the model can include numbers of entry/exit events that took place prior to a second time succeeding the first time; rates of entry/exit events expected at the second time; consumption rates of each type of surgical consumable expected at the second time; and numbers each type of surgical consumable available or consumed prior at the second time. Therefore, the computer system can generate a model (e.g., predictive model, statistical model, artificial intelligence model) that predicts future consumption rates of surgical consumables, rates of entry/exit events based on a set of surgical conditions and current consumption rates of surgical consumables, and rates of entry/exit events.

In one implementation, the computer system can generate a model that: receives as input a set of surgical conditions; and outputs the total number and type of consumables expected to be consumed during a surgical operation under the set of surgical conditions.

In one implementation, the computer system can generate a model that, based on current consumption rate of a first type of surgical consumable at a first stage of the surgical operation, predicts: the rate of consumption of the first type of surgical consumable at a second stage of the surgical operation succeeding the first stage; the rate of consumption of a second type of surgical consumable at the second stage of the surgical operation; and/or the duration of the surgical operation.

In one implementation, the computer system can generate a model that, based on current numbers and consumption rates of a surgical consumable at a first stage of the surgical operation, predicts: numbers and rates of entry/exit events.

### 8. Dynamic Prediction

Generally, at a first time during the surgical operation, the computer system can predict an out-of-stock event of a surgical consumable during the surgical operation based on the model and the current observations (e.g., rates of consumption of surgical consumables, and numbers of units of surgical consumables available). To prevent the out-of-stock event of the surgical consumable from occurring, the computer system can generate a request for re-supply of the surgical consumable and present the request to the surgical staff.

### 8.1 Phase-Based Surgical Consumable Out-of-stock Event Prediction

In one implementation, based on the model and rate of consumption of a first type of surgical consumable recorded at a first time during the surgical operation, the computer system can predict an out-of-stock event of the first type of surgical consumable at a second time succeeding the first time during the surgical operation. In particular, based on the model and rate of consumption of a first type of surgical consumable recorded at a first time during the surgical operation, the computer system can: predict a second rate of consumption of the first type of surgical consumable at the second time; and, based on the second rate of consumption and the current number of units of the first type of surgical consumable in the inventory (e.g., on the surgical supply table), predict the number of the first type of consumable in the inventory falling below a threshold at the second time. Therefore, the computer system can predict an out-of-stock event of the first type of surgical consumable at the second time during the surgical operation based on the rate of consumption of the first type of surgical consumable recorded at the first time during the surgical operation.

### 8.2 Opportunistic Request for Surgical Consumables

In one implementation, in response to predicting an out-of-stock event of a first type of surgical consumable expected at a third time during the surgical operation, the computer system can: generate a request for the first type of surgical consumable; based on the model, predict a member of the surgical staff leaving the operating room at a first time prior to the third time and returning to the operating room at a third time prior to the second time; and render the request for the first type of surgical consumable on the display at (or prior to) the first time. In this implementation, the computer system can also predict a number of units of the first type of surgical consumable utilized after the second time and prior to completion of the surgical operation. Accordingly, the request for the first type of surgical consumable rendered on the display can include the number of units of the first type of surgical consumable for the staff member to bring into operating room. Therefore, the computer system can opportunistically present a request for additional surgical consumables prior to a time when a staff member is expected to leave the operating room. Therefore, the computer system can request that the surgical consumable is resupplied without initiating an additional exit/entry event. By opportunistically presenting a request for additional surgical consumables prior to a time when a staff member is expected to leave the operating room, the computer system can reduce the risk of infection during the surgical operation by reducing the number of exit/entry events taking place during the surgical operation.

### 9.0 Pre-Operation Prediction Based on Aggregate Data

Generally, prior to the surgical operation, the computer system can, based on the model and a set of surgical conditions associated with the surgical operation, predict the total number of each type of surgical consumable expected to be consumed during the surgical operation. Therefore, the computer system can aid members of the surgical staff (e.g., circulating nurse, surgical technician) preparing the operating room for the surgical operation by informing the members of the surgical staff of the number of units of each type of surgical consumable expected to be consumed during the surgical operation.

### 9.1 Prediction Based on User Input Parameters

In one implementation, the computer system can receive user input parameters for the model from an operator (i.e., user of the computer system), the user input parameters including: user expected surgical outcome (e.g., best-case or worst-case surgical outcome); target risk to the patient; target cost of the surgical operation; and/or target completion time of the surgical operation. Prior to the surgical operation, the computer system can, based on the model and given the user input parameters and a set of surgical conditions associated with the surgical operation, predict the total number of units of each type of surgical consumable expected to be consumed during the surgical operation. In particular, the computer system can, for each type of surgical consumable and for each possible combination of surgical conditions: compute a distribution (e.g., probability distribution, histogram) of the number of units of the surgical consumable utilized during the surgical operation. Then, based on user input parameters, such as target cost of the surgical operation, the computer system can select, from the distribution, a quantity of each type of surgical consumable that corresponds to the input parameter.

For example, in response to receiving a user input parameter indicating a low target cost of the surgical operation, the computer system can, for each type of surgical consumable: select from a distribution (e.g., probability distribution, histogram) of the number of units of the surgical consumable utilized during the surgical operation, a number (e.g., output) below one standard deviation from an average (e.g., mean) number of the surgical consumable utilized. Thus, in response to receiving a user input parameter indicating a low target cost of the surgical operation, the computer system can predict lower than average quantities of surgical consumables to reduce costs of the surgical operation (as long as a probability of an out-of-stock event remains below a threshold probability).

### 10. Real-Time and Post-Operation Feedback

In one implementation, after completion of the surgical operation, the computer system can, based on the data collected during the surgical operation, compute efficiency and/or risk metrics for the surgical operation, quantities such as a percentage of time the surgeon waited for surgical delivery of consumables; a percentage of consumables indicated as waste during the surgical operation; and/or a level of risk to the patient due to total number of entry/exit events. The computer system can then: generate a notification indicating the efficiency and/or risk metrics; and present the notification to the surgical team or the operator (e.g., user of the computer system). Therefore, the computer system can provide quantitative, post-operation feedback to the surgical team and/or to the user of the computer system.

In one implementation, the computer system can provide feedback and/or warning notifications to the surgical staff during the surgical operation. For example, the computer system can display the total number of entry/exit events on the display screen during the surgical operation. Therefore, the computer system can enable the surgical staff to avoid exceeding the target number of entry/exit events during the surgical operation by providing real-time feedback during the surgical operation indicating the number of entry/exit events detected.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. The inventions as shown and/or described herein.

2. A method includes:
at a first time:
accessing a first image of a surgical field during a surgical operation; and
identifying a first quantity of unused units of a first consumable type in the first image;
at a second time:
accessing a second image of the surgical field; and
identifying a second quantity of unused units of the first consumable type in the second image
predicting a total consumption quantity of the first consumable type during the surgical operation based on a difference between the first quantity and the second quantity; and
in response to the second quantity falling below the total consumption quantity, rendering a prompt on a display, adjacent to an exit of the operating room, to retrieve a set of units of the first consumable type.
